(19) United States
(12) Patent Application Publication
(10) Pub. No.: US 2013/0065971 A1
(43) Pub. Date: Mar. 14, 2013

(54) Method for Lowering pH Using Urea Sulfate in the Presence of Corrosion Inhibitor

(71) Applicants: Gerald Stanley Rose, SR., Rock Hill, SC (US); Bobby Joel Ivey, JR., Charlotte, NC (US); Kevin Aaron Rose, Fort Mill, SC (US)

(72) Inventors: Gerald Stanley Rose, SR., Rock Hill, SC (US); Bobby Joel Ivey, JR., Charlotte, NC (US); Kevin Aaron Rose, Fort Mill, SC (US)

(73) Assignee: JSK CAPITAL LLC, Charlotte, NC (US)

(21) Appl. No.: 13/697,439

(22) PCT Filed: May 11, 2011
(86) PCT No.: PCT/US2011/036140

§ 371 Date: Nov. 12, 2012

Related U.S. Application Data
(60) Provisional application No. 61/333,851, filed on May 12, 2010.

(51) Int. Cl. C02F 1/66; A61K 47/20

(52) U.S. Cl. 514/788; 210/600

(57) ABSTRACT

A method for adjusting pH of an aqueous solution during a processing event that includes the addition of urea sulfate to the solution in the presence of a corrosion inhibitor. The processing event can be relating any number of applications, including but not limited to, textile, paper, agricultural, metals, and water applications.

### CROSS REFERENCE TO RELATED APPLICATIONS

The present application claims the benefit of priority of U.S. Provisional Application No. 61/333851, filed on May 12, 2010.

### BACKGROUND OF THE INVENTION

The present invention relates generally to pH adjusting agents, and, in particular, to a method for lowering the pH of solutions through the use of urea sulfate in the presence of a corrosion inhibitor.

The use of pH adjusting agents in solutions is known. In particular, urea sulfate has been used in textile processes to adjust the pH. This method is described in detail in U.S. Pat. No. 5,234,466, which is incorporated herein by reference in its entirety.

While the use of urea sulfate to adjust the pH of textile process solutions is effective, corrosion can and does still occur on the machinery and equipment employed in the processes. Furthermore, other applications than merely textiles can benefit from the use of pH adjusting agents, especially in a form that does cause or facilitate corrosion when used.

Accordingly, there remains a need for an improved way to adjust the pH of solutions used in numerous applications, such as paper, agricultural, pharmaceutical, metals and water applications, but that also inhibits and/or minimizes corrosion.

### SUMMARY OF THE INVENTION

The following presents a simplified summary of the invention in order to provide a basic understanding of some aspects of the invention. This summary is not an extensive overview of the invention. It is not intended to identify key or critical elements of the invention or to delineate the scope of the invention; its sole purpose is to present concepts of the invention in a simplified form as a prelude to the more detailed description that is subsequently presented.

According to its major aspects and briefly stated, the present invention includes a method for adjusting pH of an aqueous solution during a processing event that includes the addition of urea sulfate to the solution in the presence of a corrosion inhibitor. The processing event can be relating any number of applications, including but not limited to, textile, paper, agricultural, metals, and water applications.

These and other embodiments as will be realized are provided in a method for adjusting pH in an aqueous solution, comprising the steps of: providing a solution having a pH; introducing the solution to a metal; providing an amount of corrosion inhibitor; adding the corrosion inhibitor to an amount of water to form an aqueous solution; adding an amount of urea sulfate to the aqueous solution to form a pH adjusting solution; and altering said pH of said solution by adding said pH adjusting solution.

These and other embodiments as will be realized are also provided in a method for adjusting pH in an aqueous solution, comprising the steps of: providing a solution having a pH; introducing the solution to a metal; providing an amount of corrosion inhibitor and an amount of urea sulfate to an aqueous solution, wherein said aqueous solution is a pH adjusting solution; and lowering the pH of the solution by adding the pH adjusting solution.

### DETAILED DESCRIPTION OF PREFERRED EMBODIMENT

The present invention is method for adjusting the pH of processing solutions by adding urea sulfate in the presence of a corrosion inhibitor. As used herein, “corrosion inhibitor” refers to a material that reacts with a metallic surface, or the environment to which this surface is exposed, giving the surface a certain level of protection against corrosion. The mechanism of inhibition often includes a corrosion inhibitor adsorbing itself on the metallic surface, protecting the metallic surface by filing a film. Inhibitors are normally distributed from a solution or dispersion. Inhibitors slow corrosion process by either increasing the anodic or cathodic polarization behavior (Tafel slopes), reducing the movement or diffusion of ions to the metallic surface, or increasing the electrical resistance of the metallic surface.

In particular, “acid corrosion inhibition” refers to the creation of a barrier between the ionic transfer of electrons from water to metal. Therefore, any chemical or material that prevents this type of ionic transfer is a suitable acid inhibitor. The acid inhibitor chemical compounds will vary depending on the metal that needs corrosion protection. One example of an acid corrosion inhibitor is copper sulfate.

Thus, the present invention includes a method for adjusting pH of an aqueous solution during a processing event that includes the addition of urea sulfate to the solution in the presence of a corrosion inhibitor. In one embodiment, the method includes the steps of: 1) providing an aqueous processing solution; 2) introducing the aqueous solution to a metal, wherein the metal can include, but is not limited to, carbon steel or stainless steel; and 3) adjusting the pH of the aqueous processing solution with a pH adjusting solution, wherein the pH adjusting solution is prepared by the following steps: i) providing an amount of urea sulfate; ii) providing an amount of corrosion inhibitor, wherein the corrosion inhibitor can include, but is not limited to, copper sulfate or a dialkylthiourea; iii) adding the corrosion inhibitor to water or an aqueous solution; and iv) combing the amount of urea sulfate with the solution containing corrosion inhibitor to form a pH adjusting solution. The resulting solution can be employed in any number of applications, including but not limited to textile, paper, agricultural, metals, and water applications.

The particular corrosion inhibitor employed in the present invention can be any chemical or material that creating a barrier between the ionic transfer of electrons from water to metal. However, based on the properties of the metals in which corrosion is being inhibited, certain materials are preferred to others. For example, if the metal at issue is carbon steel, a preferred corrosion inhibitor includes dialkyithioureas, such as diethylthiourea and dibutylthiourea. If the metal is stainless steel, a preferred corrosion inhibitor includes cupric sulfate or copper sulfate, as well as commercially available materials or blends of materials effective at inhibiting corrosion, including but not limited to a material from the Rodine family, such as Rodine 214, Rodine 213, Rodine 145L, Rodine 103, Rodine 102; Rodine 95, and Rodine 85, Rodine 55, Rodine 50, Rodine 31A, etc.; from the Dodicor family, such as Dodicor 2565 and Dodicor 2725 NF, Dodicor V 5277, Dodicor V 4712, Dodicor 1830, Dodicor 4543, Dodicor 3747, etc.; or from the Armohib family, such as Armohib Cl-219, Armohib 209, Armohib 31, Armohib 28, etc. These blends can also work to inhibit corrosion of carbon steel type metals. It is believed that Rodine inhibitors are provided by JMN Specialties, Inc. and Henkel Corp., Dodicor inhibitors are provided by Clariant, and Armohib inhibitors are provided by Akzo Nobel Surfactants LLC.

In another embodiment, the present invention includes a method for adjusting the pH of an aqueous solution including the following steps: 1) providing an aqueous processing solution; 2) introducing the processing solution to a metal, the metal being carbon steel; and 3) adjusting the pH of the aqueous processing solution by introducing a pH adjusting solution, wherein the pH adjusting solution is prepared by the following steps: i) providing an effective amount of dialkylthiourea selected from diethylthiourea or dibutylthiourea; ii) adding the dialkylthiourea to an amount of water so that the aqueous solution contains between about 10 ppm to about 1000 ppm of dialkylthiourea; and iii) adding an effective amount of urea sulfate to the aqueous solution to form the pH adjusting solution.

The urea sulfate can be formed from a ratio of between approximately 1:4 and 4:1 moles of urea to sulfuric acid. Alternatively, the urea sulfate can be formed from a molar ratio of urea to sulfuric acid of between about 2.5 and about 0.25 moles of urea to sulfuric acid. Alternatively, the urea sulfate can be formed from a molar ratio of urea to sulfuric acid of about 1 mole to about 1 mole of sulfuric acid.

In another embodiment, the pH adjusting solution is prepared by the following steps: 1) providing an effective amount of dialkylthiourea; 2) adding the effective amount of dialkylthiourea to an amount of water so that the acqueous solution contains about 200 ppm of dialkylthiourea; and 3) adding an effective amount of urea sulfate to the solution to form the pH adjusting solution.

In yet another embodiment, the present invention includes a method for adjusting the pH of an aqueous solution including the following steps: 1) providing an aqueous processing solution; 2) introducing the processing solution to a metal, the metal being stainless steel; and 3) adjusting the pH of the aqueous processing solution by introducing a pH adjusting solution, wherein the pH adjusting solution is prepared by the following steps: i) providing an effective amount of copper sulfate; ii) adding the copper sulfate to an amount of water so that the aqueous solution contains between about 10 ppm to about 1000 ppm of copper sulfate; and iii) adding an effective amount of urea sulfate to the aqueous solution to form the pH adjusting solution.

The urea sulfate can be formed from a ratio of between approximately 1:4 and 4:1 moles of urea to sulfuric acid. Alternatively, the urea sulfate can be formed from a molar ratio of urea to sulfuric acid of between about 2.5 and about 0.25 moles of urea to sulfuric acid. Alternatively, the urea sulfate can be formed from a molar ratio of urea to sulfuric acid of about 1 mole to about 1 mole of sulfuric acid.

In another embodiment, the pH adjusting solution is prepared by the following steps: 1) providing an effective amount of copper sulfate; 2) adding the effective amount of copper sulfate to an amount of water so that the aqueous solution contains about 200 ppm of copper sulfate; and 3) adding an effective amount of urea sulfate to the aqueous solution to form the pH adjusting solution. The urea sulfate can be formed from a molar ratio of urea to sulfuric acid of between about 2.5 and about 0.25 moles of urea to sulfuric acid.

A listing of results obtained from testing various corrosion inhibitors is shown below. The products were tested using a standard NACE-TM0169-76 method, which is incorporated herein in its entirety, on carbon steel coupons (1010).

The results shown in mils per year (mpy) are as follows:

TABLE 1

| | 100% concentrate | | 20% concentrate | |
|---|---|---|---|---|
| Ultra Biocid | 51 | mpy | 1039.5 | mpy |
| Ultra Biocid EH | 21 | mpy | 21.1 | mpy |
| Acetic Acid | 748 | mpy | 81.5 | mpy |
| Citric Acid | 1022 | mpy | 112.3 | mpy |

The term “Ultra Biocid” refers to an aqueous solution having 99% Urea sulfate and 1% copper sulfate. The term “Ultra Biocid EH” refers to an aqueous solution containing 99% urea sulfate and 1% Armohib, which is a blend containing copper sulfate. Ultra Biocid and Ultra Biocid EH are presently commercially available and sold by Ultra-chem Industries, Inc. In table 1, “100% Concentrate” refers to material which is used as purchased and “20% Concentrate” refers to material diluted to 20% with the addition of water.

The data shown in the table above indicates that certain corrosion inhibitors are more effective at preventing corrosion when used with certain metal. Moreover, without any corrosion inhibitor, the corrosion is made even worse, as shown by the mpy values of acetic acid and citric acid.

Thus, without the proper inhibitor the corrosion as measured by mpy's is severe. For example, Utra Biocid inhibits against corrosion more effectively when the metal is a stainless steel 316 type. Though not shown, the results above for the Ultra Biocid and Ultra Biocid EH become approximately reversed if the metal used is stainless steel.

The Ultra Biocid EH inhibits corrosion against carbon steel type products better than Ultra Biocid, as shown by the low mpy value at merely 20% concentrate of Ultra Biocid EH. The addition of water to Ultra Biocid in the presence of carbon steel, therefore, greatly increases corrosion rates. The addition of water to Ultra Biocid EH does not increase its corrosion rates. Nevertheless, at 100% concentrate, Ultra Biocid still operates to inhibit corrosion. The 100% concentrated Ultra Biocid and Ultra Biocid EH has very low corrosion characteristics in the absence of water.

Those skilled in the art of pH adjusting agents and the chemical arts will appreciate from the foregoing description of preferred embodiments that substitutions and modification can be made without departing from the spirit and scope of the invention which is defined by the appended claims.

What is claimed is:

1. A method for adjusting pH in an aqueous solution, comprising the steps of:
providing a solution having a pH;
introducing said solution to a metal;
providing an amount of corrosion inhibitor;
adding said corrosion inhibitor to an amount of water to form an aqueous solution;
adding an amount of urea sulfate to said aqueous solution to form a pH adjusting solution; and
altering said pH of said solution by adding said pH adjusting solution.

2. The method as recited in claim 1, wherein said corrosion inhibitor is copper sulfate.

3. The method as recited in claim 1, wherein said amount of corrosion inhibitor is between about 10 ppm and about 2000 ppm.

4. The method as recited in claim 1, wherein said amount of corrosion inhibitor is about 200 ppm.

5. The method as recited in claim 1, wherein said metal is carbon steel.

6. The method as recited in claim 5, wherein said corrosion inhibitor is a dialkylthiourea.

7. The method as recited in claim 6, wherein said dialkylthioureas is selected from diethylthiourea or dibutlethiourea.

8. The method as recited in claim 5, wherein said pH adjusting solution is Ultra Biocid EH.

9. The method as recited in claim 8, wherein said Ultra Biocid EH is 99% urea sulfate and 1% Armohib.

10. The method as recited in claim 1, wherein said metal is stainless steel.

11. The method as recited in claim 10, wherein said corrosion inhibitor is copper sulfate.

12. The method as recited in claim 10, wherein said corrosion inhibitor is Rodine 214, Rodine 213, Rodine 145L, Rodine 103, Rodine 102, Rodine 95, Rodine 85, Rodine 55, Rodine 50, Rodine 31A, Dodicor 2565, Dodicor 2725 NF, Dodicor V 5277, Dodicor V 4712, Dodicor 1830, Dodicor 4543, Dodicor 3747, Armohib CI-219, Armohib 209, Armohib 31, or Armohib 28.

13. The method as recited in claim 10, wherein said pH adjusting solution is Ultra Biocid.

14. The method as recited in claim 13, wherein said Ultra Biocid is 99% urea sulfate and 1% copper sulfate.

15. The method as recited in claim 1, wherein said solution is an application selected from textiles, paper, agricultural, pharmaceutical, metals or water.

16. The method of claim 1, wherein said urea sulfate is formed from a molar ratio of urea to sulfuric acid of between about 2.5 and about 0.25 moles of urea to 1 mole of sulfuric acid.

17. The method of claim 1, wherein said urea sulfate is formed from an approximate 1:1 molar ratio of urea to sulfuric acid.

18. The method of claim 1, wherein said urea sulfate is formed from a ratio of between approximately 1:4 and 4:1 moles of urea to sulfuric acid.

19. A method for adjusting pH in an aqueous solution, comprising the steps of:
providing a solution having a pH;
introducing said solution to a metal;
providing an amount of corrosion inhibitor and an amount of urea sulfate to an aqueous solution, wherein said aqueous solution is a pH adjusting solution; and
lowering said pH of said solution by adding said pH adjusting solution.

20. The method as recited in claim 19, wherein said corrosion inhibitor is selected from copper sulfate, dialkylthiourea, Rodine 214, Rodine 213, Rodine 145L, Rodine 103, Rodine 102, Rodine 95, Rodine 85, Rodine 55, Rodine 50, Rodine 31A, Dodicor 2565, Dodicor 2725 NF, Dodicor V 5277, Dodicor V 4712, Dodicor 1830, Dodicor 4543, Dodicor 3747, Armohib CI-219, Armohib 209, Armohib 31, or Armohib 28.
